(19) United States
(12) Patent Application Publication
(10) Pub. No.: US 2003/0159230 A1
(43) Pub. Date: Aug. 28, 2003

(54) Upright-type vacuum cleaner

(76) Inventor: Jang-keun Oh, Gwangju-city (KR)

Correspondence Address: Todd S. Parkhurst, Holland & Knight LLP, Suite 800, 55 West Monroe Street, Chicago, IL, 60603-5144, US

(21) Appl. No.: 10/211,694

(22) Filed: Aug. 2, 2002

(30) Foreign Application Priority Data
Feb. 28, 2002 (KR) 2002-10797

(51) Int. Cl. A47L 11/29

(52) U.S. Cl. 15/320

(57) ABSTRACT

An upright-type vacuum cleaner has a cleaner body having a driving unit to generate a suction force, and a filth-collecting chamber to collect filth drawn by the suction of the driving unit; a suction brush unit disposed at a lower part of the cleaner body, the suction brush unit operating to draw the filth from a cleaning surface by the suction force of the driving unit; and an electrolytic liquid supplying apparatus disposed in the cleaner body to spray electrolytic liquid on the cleaning surface through a bottom of the suction brush unit.

### CLAIM OF PRIORITY

This application hereby refers to, and incorporates herein by reference, an earlier filed patent application entitled UPRIGHT-TYPE VACUUM CLEANER, filed in the Korean Industrial Property Office on Feb. 28, 2002, and there duly assigned Serial Number 2002-10797. Applicant hereby claims all benefits accruing under 35 U. S. C. Section 119 for and from said earlier filed Korean patent application.

### BACKGROUND OF THE INVENTION

1. Field of the Invention

The present invention relates to an upright-type vacuum cleaner, and more particularly, to an upright-type vacuum cleaner having an apparatus for generating electrolytic liquid.

2. Description of the Prior Art

A conventional upright-type vacuum cleaner includes a cleaner body having a driving motor to generate suction force, and a suction brush rotatably disposed at a lower part of the cleaner body.

The cleaner body has a filtering means and a filth-collecting chamber installed therein. Filth drawn through the suction brush by the suction force of the driving motor is filtered by the filtering means is and collected in the filth-collecting chamber.

A handle has an on/off switch disposed at an upper part of the cleaner body. A user moves the cleaner over a floor, carpet or other surface to be vacuum cleaned by pushing and pulling the handle, and the filth on the floors, carpet or other surface is drawn and removed from the surface.

However, some filth adhering to the floor or carpet cannot be easily removed by the suction alone. Thus, a rotating suction brush is installed to remove the stubborn dirt on the floor or the carpet.

In addition, to get rid of filth that is not easily removed by the rotating brush, detergent is supplied to the bottom of the suction brush.

In the conventional upright-type vacuum cleaner, the detergent must be diluted in water. Accordingly, the vacuum cleaner is not convenient to use and the operating cost is high.

Furthermore, a detergent feeding path can be polluted or clogged by the residue of the detergent.

### SUMMARY OF THE INVENTION

The present invention has been made to overcome these problems of the prior art. Accordingly, it is the object of the present invention to provide an upright-type vacuum cleaner having an improved structure to clean stubborn dirt on the floor or a carpet without using a detergent.

To accomplish these objectives, we provide an upright-type vacuum cleaner according to the present invention, comprising a cleaner body having a driving unit to generate a suction force and a filth-collecting chamber to collect the filth drawn by the suction force of the driving unit; a suction brush unit disposed at a lower part of the cleaner body, the suction brush unit acting to draw away the filth on a cleaning surface by the suction force of the driving unit; and an electrolytic liquid supplying apparatus disposed in the cleaner body in order to spray electrolytic liquid to the cleaning surface through a bottom of the suction brush unit.

It is preferable that the electrolytic liquid supplying apparatus includes an electrolytic liquid generation unit installed in the cleaner body to generate the electrolytic liquid from supplied water; and a spraying unit to spray the electrolytic liquid generated at the electrolytic liquid generation unit to a location near the air suction port of the suction brush.

It is advisable that the spraying unit includes an electrolytic liquid transferring path to connect the electrolytic liquid generation unit and the air suction port of the suction brush; a spray pump on the electrolytic liquid transferring path; and a spray nozzle disposed at an end of the electrolytic liquid transferring path in order to spray the electrolytic liquid transferred to a location near the air suction port of the suction brush.

### BRIEF DESCRIPTION OF THE DRAWINGS

The above-mentioned object and the feature of the present invention will be more apparent by reading the preferred embodiment of the present invention and by reference to the appended drawings, in which:

FIG. 1 is a partially cut-away view schematically showing an upright-type vacuum cleaner according to the present invention; and

FIG. 2 is a block diagram schematically showing an electrolytic liquid generation unit provided in the vacuum cleaner of FIG. 1.

### DESCRIPTION OF THE PREFERRED EMBODIMENT

Hereinbelow, an upright-type vacuum cleaner according to the preferred embodiment of the present invention will be described in greater detail by referring to the appended drawings.

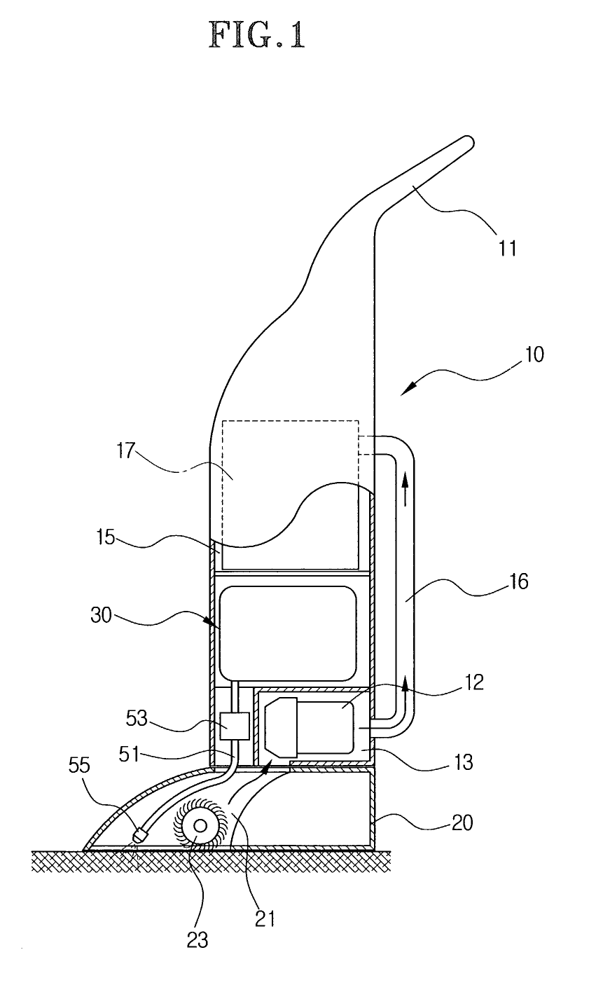

Referring to FIG. 1, the upright-type vacuum cleaner according to the preferred embodiment of the present invention comprises a cleaner body 10, a suction brush unit 20 movably connected with a lower part of the cleaner body 10, and an electrolytic liquid supplying apparatus 30 installed at the cleaner body 10.

A handle 11 is disposed at an upper part of the cleaner body 10. A driving unit 13 and a filth-collecting chamber 15 are grooved inside of the cleaner body 10. A driving motor 12 to supply a suction force is installed inside the driving unit 13. The driving unit 13 is connected with the filth-collecting chamber 15 through an air passage 16.

A filth container 17 to collect filth included in drawn air is installed inside the filth-collecting chamber 15. The filth container 17 can have a filter (not shown) therein. Moreover, a well-known cyclone filth-separating apparatus can be included in the filth-collecting chamber 15. Additionally, a liquid separation means can be installed in the filth-collecting chamber 15, to separately collect dirty liquid when the liquid is drawn through the suction brush unit 20. The above elements are all generally well known, and so detailed descriptions are omitted here.

The suction brush unit 20 is installed at a lower part of the cleaner body 10. The suction brush unit 20 has an air suction port 21 to draw the filth from a dirty surface. The air suction port 21 is connected to the driving unit 13. Moreover, a rotation brush 23 can be further disposed at the open and/or bottom of the air suction port 21. The rotation brush 23 cleans the cleaning surface by being rotated by the suction force of the driving motor 13 and by heating the surface.

The electrolytic liquid supplying apparatus 30 is for cleaning stubborn dirt from a carpet or floor, and supplies electrolytic liquid through the suction brush unit 20. The electrolytic liquid supplying apparatus 30 includes an electrolytic liquid generation unit 31 installed inside the cleaner body 10 and a spraying unit 33 to spray electrolytic liquid generated at the electrolytic liquid generation unit 31 on the cleaning surface at or near the air suction port 21 of the suction brush 20.

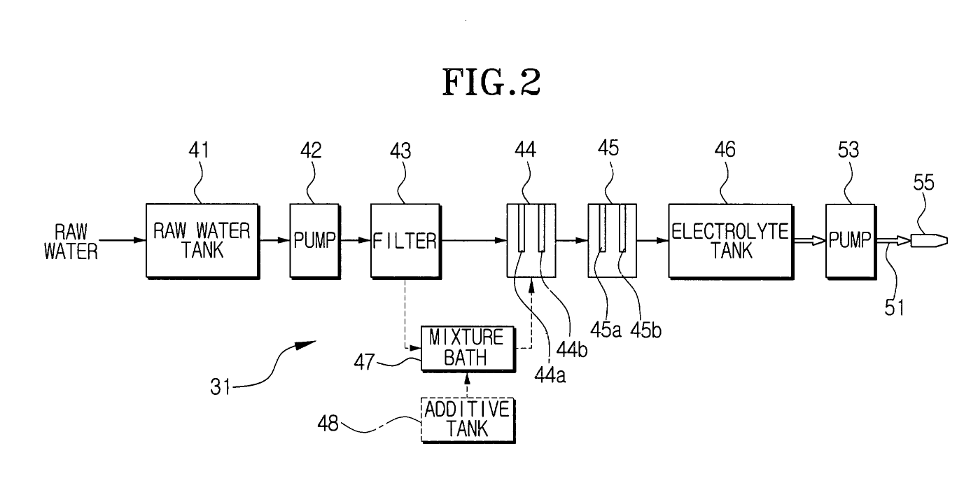

One example of the electrolytic liquid generation unit 31 is shown in FIG. 2. The electrolytic liquid generation unit 31 comprises a water tank 41 to store water, like tap water, fresh water or well water, a pump 42 to transfer the water in the water tank 41, a filter 43 to filter the water passed through the pump 42, a first electrolytic cell 44, a second electrolytic cell 45, and an electrolytic liquid tank 46.

The filter 43 filters ions or impurities from the water. The water passed through the filter 43 flows into the first electrolytic cell 44. The first electrolytic cell 44 has a cathode plate 44a and an anode plate 44b. The first electrolytic cell 44 electrolyzes the flowing water and transfers the water to the second electrolytic cell 45. The second electrolytic cell 45 also has a cathode plate 45a and an anode plate 45b. The second electrolytic cell 45 generates electrolytic liquid by further electrolyzing that water which has not been electrolyzed at the first electrolytic cell 44.

During operation, the first electrolytic cell 44 and the second electrolytic cell 45 generate gas (mainly hydrogen gas). To get rid of the gas, a gas disposal system of known construction can be provided.

The electrolytic liquid generated at the second electrolytic cell 45 is transferred to the electrolytic liquid holding tank 46 to be stored.

A mixture tank 47 to temporarily store the water passed through the filter 43 can be provided. A catalyzer material is supplied from a separately disposed additive tank 48, and the catalyzer is mixed with the water in the mixture tank 47. Here, sodium chloride, potassium chloride, calcium chloride can be used as the catalyzer. The catalyzer can be selectively used.

As suggested in FIG. 2, the liquid mixture and catalyzer in the mixture tank 47 can be transferred to the first electrolytic cell 44 and electrolyzed.

The above-described electrolytic liquid generation unit 31 is one example of several well-known electrolytic liquid generating means, and other various examples can be available.

As illustrated in FIG. 1, the spraying unit 33 has an electrolytic liquid transferring tube or path 51 to connect the electrolytic liquid tank 46 and the air suction port 21 of the suction brush unit 20. A spray pump 53 is disposed on the electrolytic liquid transferring path 51, and a spray nozzle 55 disposed at an end of the electrolytic liquid transferring path 51. The spray pump 53 pumps the electrolytic liquid stored in the electrolytic liquid tank 46 and forces the electrolytic liquid through the spray nozzle 55.

In operation, the upright-type vacuum cleaner having the electrolytic liquid supplying apparatus according to the preferred embodiment of the present invention having the above structure sprays electrolytic liquid generated at the electrolytic liquid supplying apparatus 30 on the carpet or floor and thereafter draws the filth and water through the air suction port 21 of the suction brush unit 20. The stubborn dirt is effectively removed by the sprayed electrolytic liquid. Here, the electrolytic liquid is generally used as waste-water treatment, bleaching agent for cotton fabrics and antiseptic/sanitizer for a water supply. The electrolytic liquid can effectively clean adhering filth without adding separate detergent.

Accordingly, there is no need to dissolve detergent in the water. Moreover, detergent is not required, and so costs are reduced.

Although the preferred embodiment of the present invention has been described, it will be understood by those skilled in the art that the present invention should not be limited to the described preferred embodiment, but various changes and modifications can be made within the spirit and the scope of the present invention. Accordingly, the scope of the present invention is not limited within the described range but is defined by the following claims.

What is claimed is:

1. An upright-type vacuum cleaner comprising:
a cleaner body having a driving unit to generate a suction force and a filth-collecting chamber to collect a filth drawn by the suction force generated by the driving unit;
a suction brush unit disposed at a lower part of the cleaner body, the suction brush unit operating to draw the filth from a cleaning surface by the suction force of the driving unit; and
an electrolytic liquid supplying apparatus disposed in the cleaner body for spraying electrolytic liquid on the cleaning surface through a bottom of the suction brush unit.

2. The upright-type vacuum cleaner of claim 1 wherein the electrolytic liquid supplying apparatus includes:
an electrolytic liquid generation unit installed in the cleaner body to generate the electrolytic liquid from supplied water; and
a spraying unit to spray the electrolytic liquid generated at the electrolytic liquid generation unit to an air suction port of the suction brush unit.

3. The upright-type vacuum cleaner of claim 2 wherein the spraying unit includes:
an electrolytic liquid transferring path to connect the electrolytic liquid generation unit and the air suction port of the suction brush unit;
a spraying pump disposed in the electrolytic liquid transferring path; and
a spray nozzle disposed at an end of the electrolytic liquid transferring path to spray the electrolytic liquid to a point near the air suction port of the suction brush unit.
